(19) United States
(12) Patent Application Publication
(10) Pub. No.: US 2015/0065834 A1
(43) Pub. Date: Mar. 5, 2015

(54) INTEGRATED LANCING DEVICE WITH TESTING METER

(71) Applicant: KHALDOUN FAHAD SULAIMAN AL SAHLI, Kuwait (KW)

(72) Inventor: KHALDOUN FAHAD SULAIMAN AL SAHLI, Kuwait (KW)

(21) Appl. No.: 14/065,884

(22) Filed: Oct. 29, 2013

(30) Foreign Application Priority Data
Aug. 27, 2013 (GB) GB1315190.7

(51) Int. Cl. A61B 5/151; A61B 5/157

(52) U.S. Cl. CPC A61B 5/15101; A61B 5/15146; A61B 5/157
USPC 600/365

(57) ABSTRACT

The medical testing instrument integrates the multiclix insulin pen with lancet and with testing meter which show the result on the touch screen. The device also, can connect to smart phone application through Bluetooth or other wireless connections.

### BACKGROUND OF THE INVENTION

1. Field of the Invention

In general, this invention relates to skin lancing devices, analyte sensors and analysis meters for determining biological analyte levels, and more specifically, a portable device that integrates lancing device in shape of insulin pen with test meter.

2. Description of the Related Art

Methods and devices used by a patient to measure a bioanalyte are well known in the art. For example, currently available technology allows a diabetic patient to monitor his own blood glucose level by drawing a blood sample with a lancing device, using an electrochemical sensor strip to capture the blood sample, and using an electronic meter connected to the sensor strip to analyze the blood sample and display the result. Until recently, relatively large sample volumes were required to be drawn, generally 3 microliters or more of blood or other biological fluid. These fluid samples are obtained from a patient, for example, using a needle and syringe, or by lancing a portion of the skin such as the fingertip and “milking” the area to obtain a useful sample volume. These procedures are inconvenient for the patient, and often painful, particularly when frequent samples are required. Less painful methods for obtaining a sample are known such as lancing the arm or thigh, which have lower nerve ending density. However, lancing the body in these preferred regions typically produces submicroliter samples of blood, because these regions are not heavily supplied with near-surface capillary vessels. The recently introduced FreeStyle™ Blood Glucose Monitoring System developed by TheraSense, Inc. of Alameda, Calif., is capable of consistently, accurately and precisely measuring sample sizes of only [⅓] microliter using this preferred “alternate site testing” (AST). U.S. Pat. No. 6,299,757, issued Oct. 9, 2001 to TheraSense, Inc. and incorporated herein by reference describes the construction and operation of the above FreeStyle system. U.S. Pat. No. 6,283,982 issued Sep. 4, 2001 to TheraSense, Inc. and incorporated herein by reference describes a lancing device that is used in the FreeStyle system.

U.S. Pat. No. US2012083679 is directed to an integrated device for sampling and testing an analyte. The device generally comprises a housing, a lancing device for sampling an analyte, a test strip for substantially capturing at least a portion of the analyte, and a display unit for displaying a result corresponding to the captured portion of the analyte. The invention is further directed to methods for sampling and testing. For example, one method comprises performing a single operation to sample an analyte, to capture the sampled analyte, to perform testing on the sampled analyte, and to display a result corresponding to the performed test. A method such as this can be carried out using an integrated sampling and testing device of the invention, for example, by placing the device the device on a test site of a subject, such as a patient, and performing the single operation to obtain a test result. The invention has particular application in the sampling and testing of analytes in blood, such as the blood of a diabetic patient.

EP patent No. EP2213231A1 An integrated medical testing device includes a testing meter and lancing device, the meter having a housing, a display, a test element opening, a firing mechanism and a navigation mechanism integrated with a lancing device. The lancing device is at least partially located in the housing and includes a tip having an opening through which a lancet extends upon firing. Various mechanisms can be used to extend the tip from the housing and to prime and fire the lancing device. The lancing device can accommodate a single lancet or a caatidge having multiple lancets. If a cartridge is used, various mechanisms can be utilized to position an unused lancet for firing after a test has been conducted. The relative locations of the lancing device and test element opening are such that the integrated meter and lancing device can be used with a test element inserted in the test element opening. The integrated meter and lancing device can include status indicators that reflect the state of the lancing device.

ACCU-CHEK® Multiclix lancing device Reduce the pain and hassle of blood glucose testing. The ACCU-CHEK Multiclix lancing device uses a drum of 6 preloaded lancets. Plus, it uses the same technology as the ACCU-CHEK FastClix device, which has been proven least painful. 1 With 11 depth settings for maximum comfort, and minimal side-to-side motion for less skin tearing, it helps eliminate the pain of blood sugar checks.

What is needed and has not been provided by the prior art is a simpler testing method using a compact, unitary testing device.

### SUMMARY OF THE INVENTION

The testing instrument of the present invention provides a method for obtaining a sample and testing that sample using a single device. Further, the instrument automatically performs all the testing steps in the proper order with the proper delays for each. The entire testing process is initiated by the patient with a single press of a button. The instrument automatically inserts and retracts a lancet into the skin with the proper speed and force, waits a predetermined time for a fluid sample to form on the skin, aligns the fill channel of a test strip with the small fluid sample and brings the two into contact to capture the sample, indicates to the patient when a sufficient sample has been captured, waits for electrochemical testing of the sample to be complete, displays the test result to the patient, and records all of the test results for later review, analysis and/or uploading to a computer network.

The medical testing instrument integrates the multiclix insulin pen with lancet and with testing meter which show the result on the touch screen. The device also, can connect to smart phone application through Bluetooth or other wireless connections.

### BRIEF DESCRIPTION OF THE DRAWINGS

FIG. 1 is an isometric view of the integrated medical testing insulin pen.

FIG. 2 is an isometric view of the integrated medical testing insulin pen two main parts.

FIG. 3 is an environmental, outside detailed perspective view of the integrated medical testing insulin pen.

FIG. 4 is an environmental, inside detailed perspective view of the integrated medical testing insulin pen.

FIG. 5 is an environmental, inside detailed perspective view of the integrated medical testing insulin pen from the other side.

FIG. 6 is an environmental, more inside detailed perspective view of the integrated medical testing insulin pen.

FIG. 7 is an environmental, more inside detailed perspective view of the integrated medical testing insulin pen.

FIG. 8 is an environmental, perspective view of separate inside parts of the integrated medical testing insulin pen.

FIG. 9 is an environmental, perspective view of the disposal unit of (sprit and lancet container) of the integrated medical testing insulin pen.

FIG. 10 is an environmental, perspective view of lancet of the integrated medical testing insulin pen.

FIG. 11 is an environmental, more inside details perspective view of lancet pusher of the integrated medical testing insulin pen.

FIG. 12 is an environmental, more inside details of lancet holder and launcher perspective view of the integrated medical testing insulin pen.

FIG. 13 is an environmental, perspective view of electronic circuit board of the integrated medical testing insulin pen.

FIG. 14 is an environmental, more details perspective view of the integrated medical testing insulin pen.

FIG. 15 is an environmental, more details perspective view of the integrated medical testing insulin pen.

FIG. 16 is an environmental, more details perspective view of the integrated medical testing insulin pen.

FIGS. 17 to 23 is an environmental, collection of more details perspective view of the integrated medical testing insulin pen.

Similar reference characters denote corresponding features consistently throughout the attached drawings.

### DETAILED DESCRIPTION OF THE INVENTION

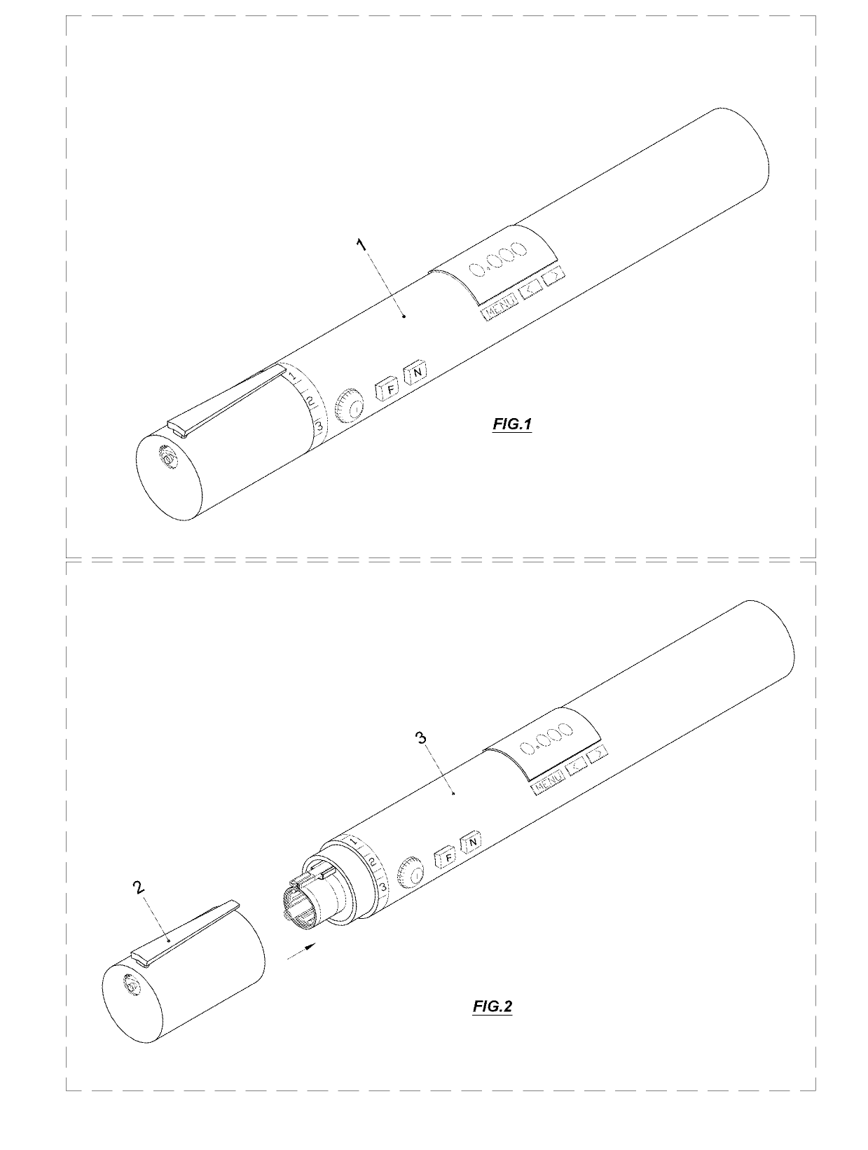

Referring to FIG. 1a and 2, is an integrated medical sampling and testing insulin pen device 1 has a pen cap 2 attached to a housing 3 which contains lancing device, testing meter, mechanical and electric gearbox, and control circuit plates.

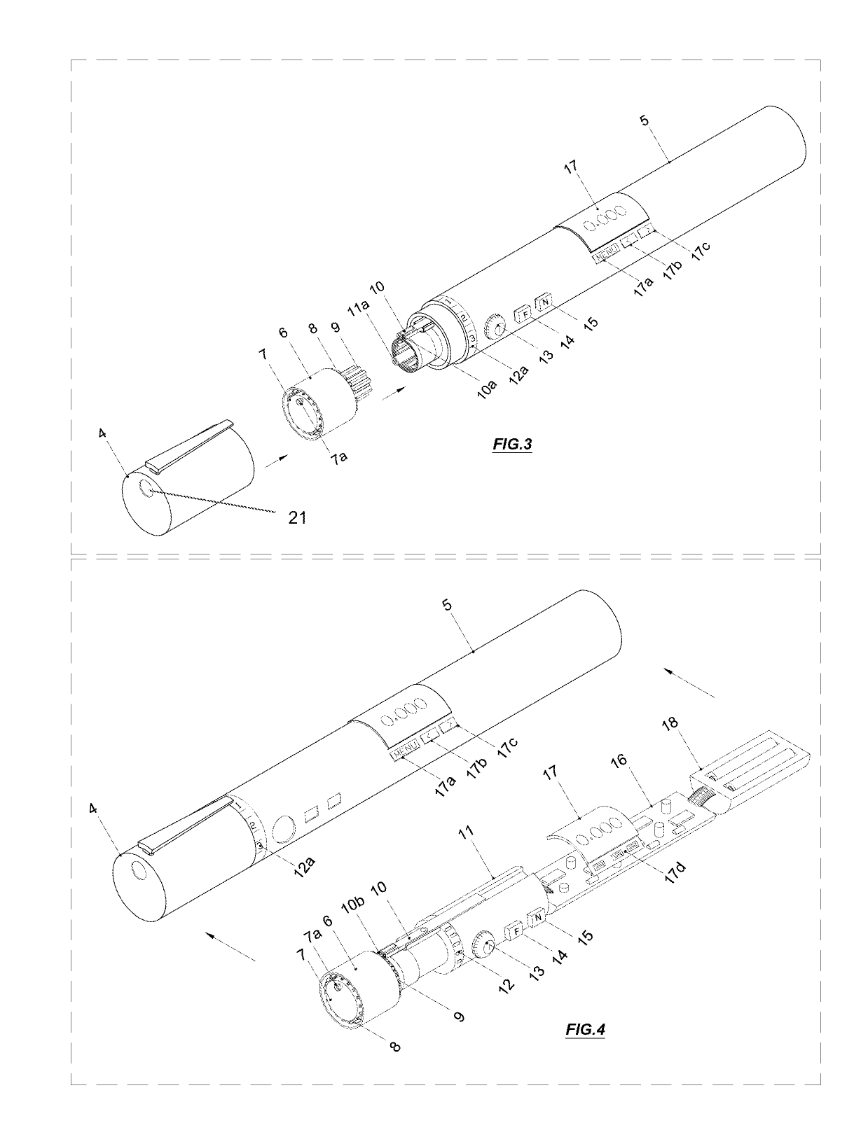

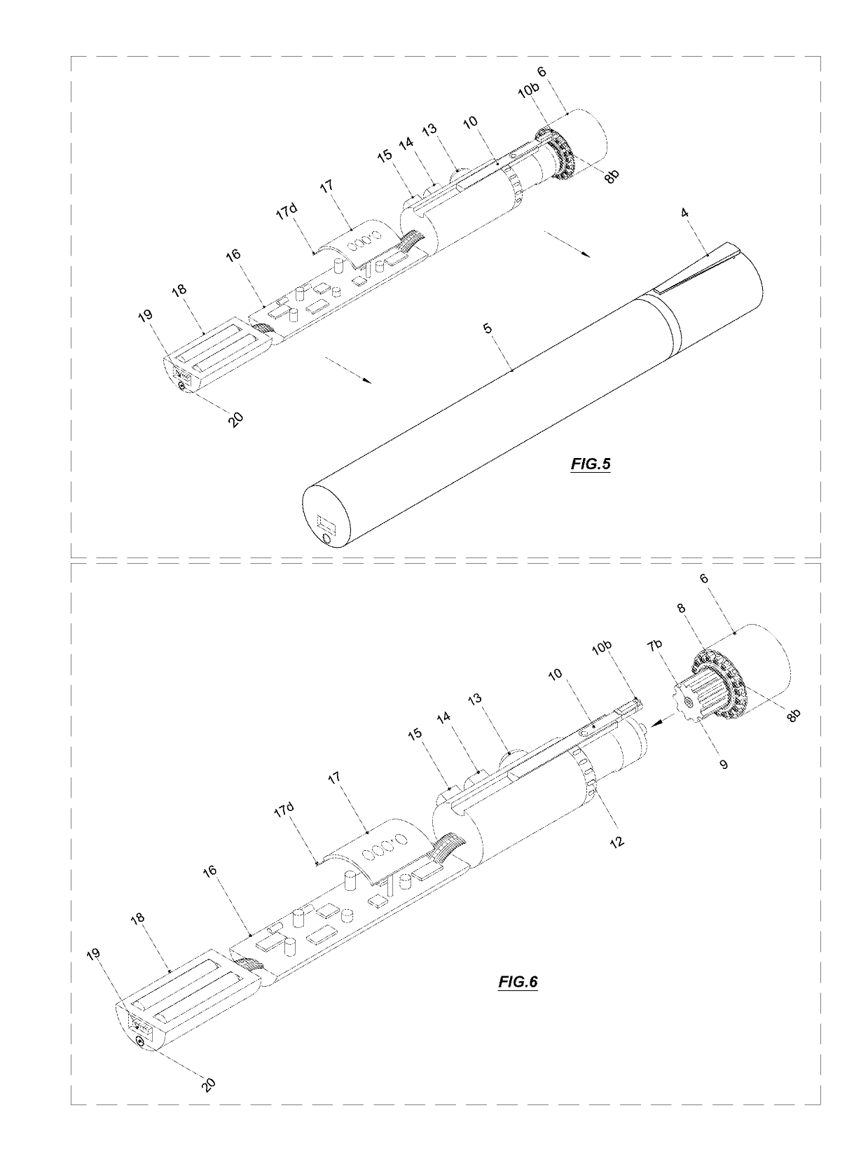

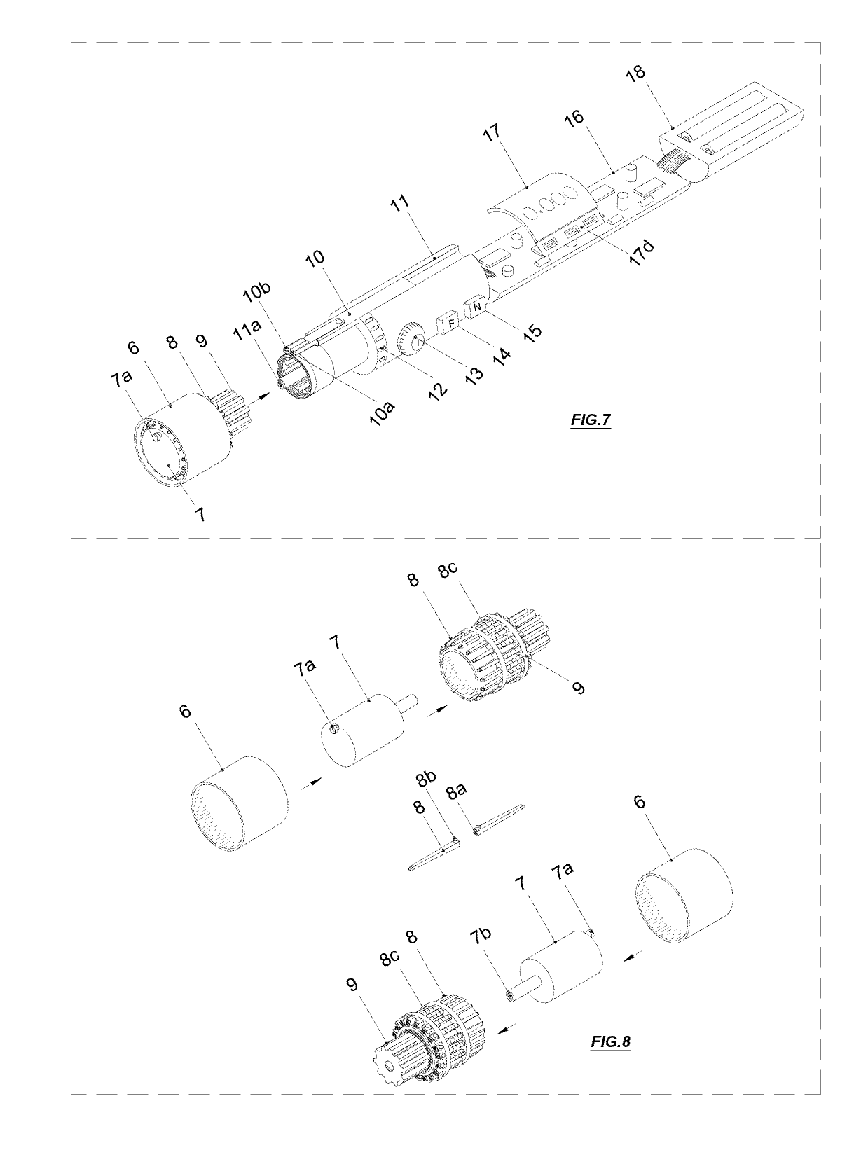

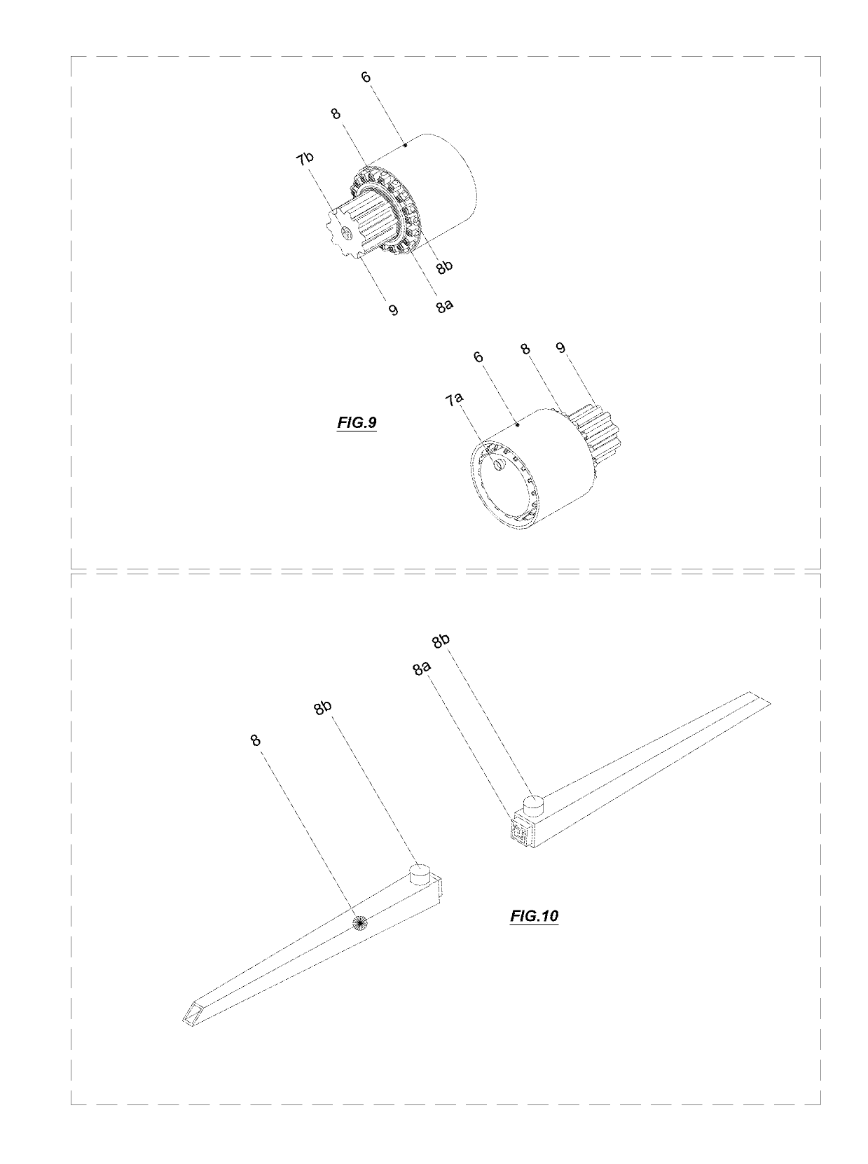

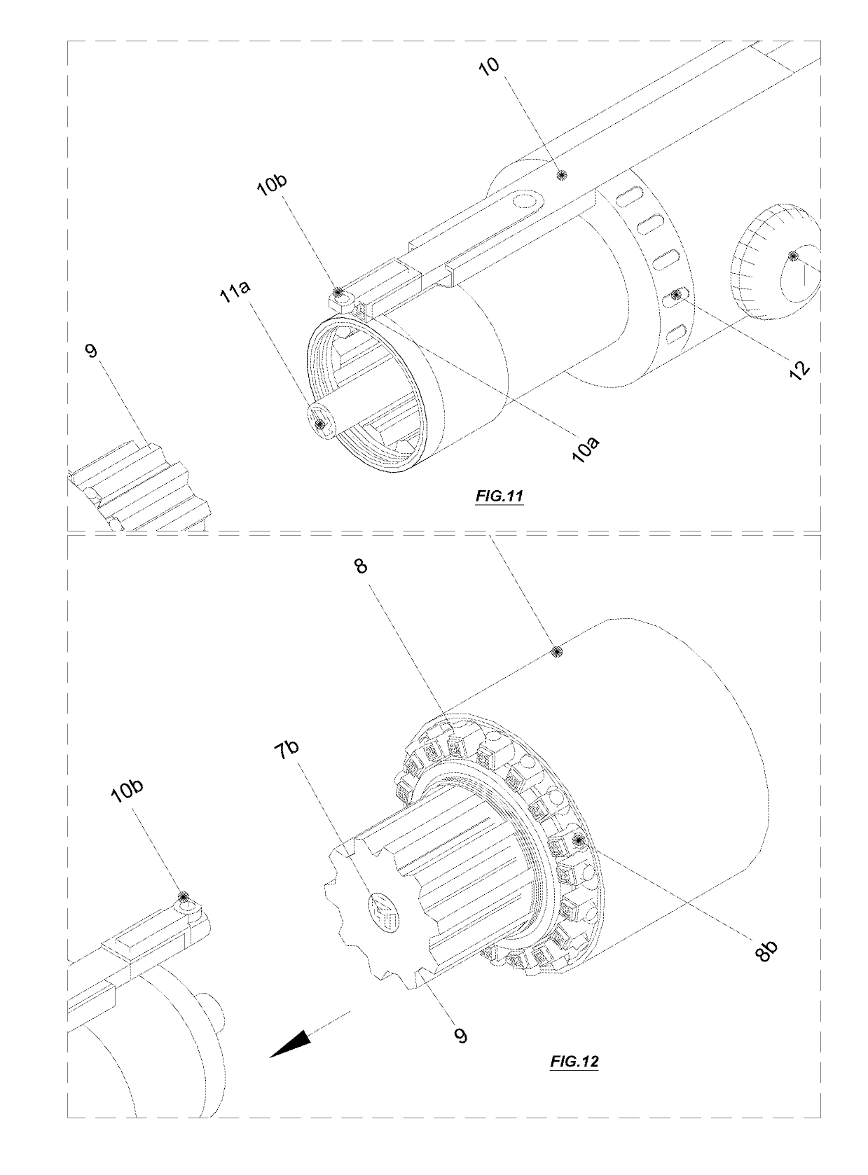

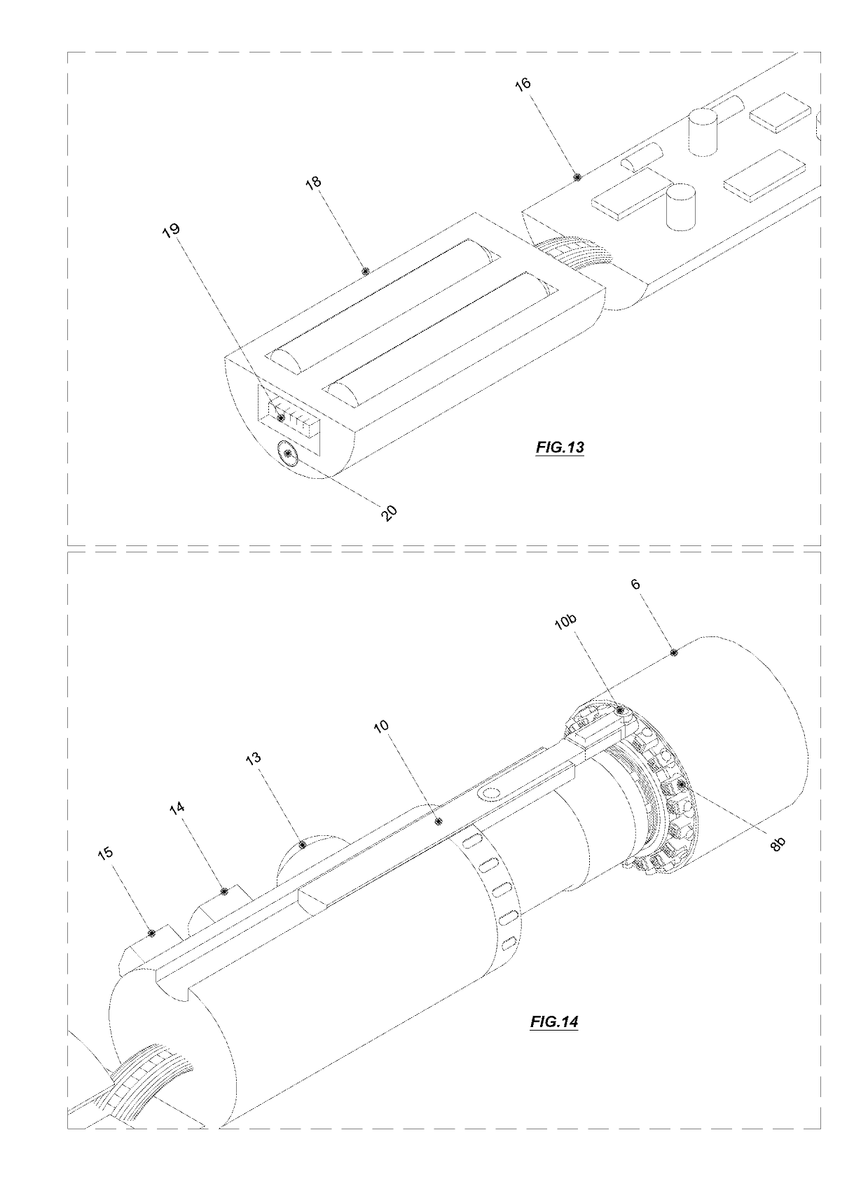

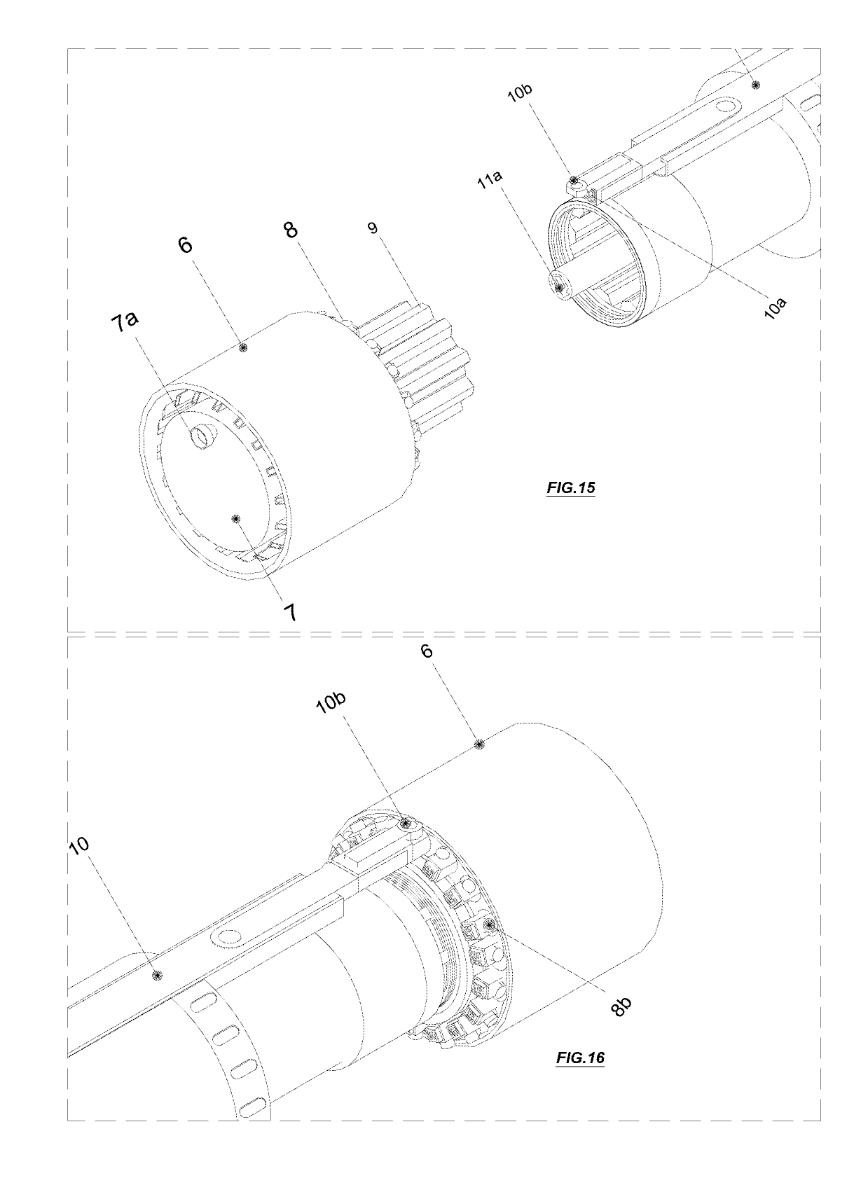

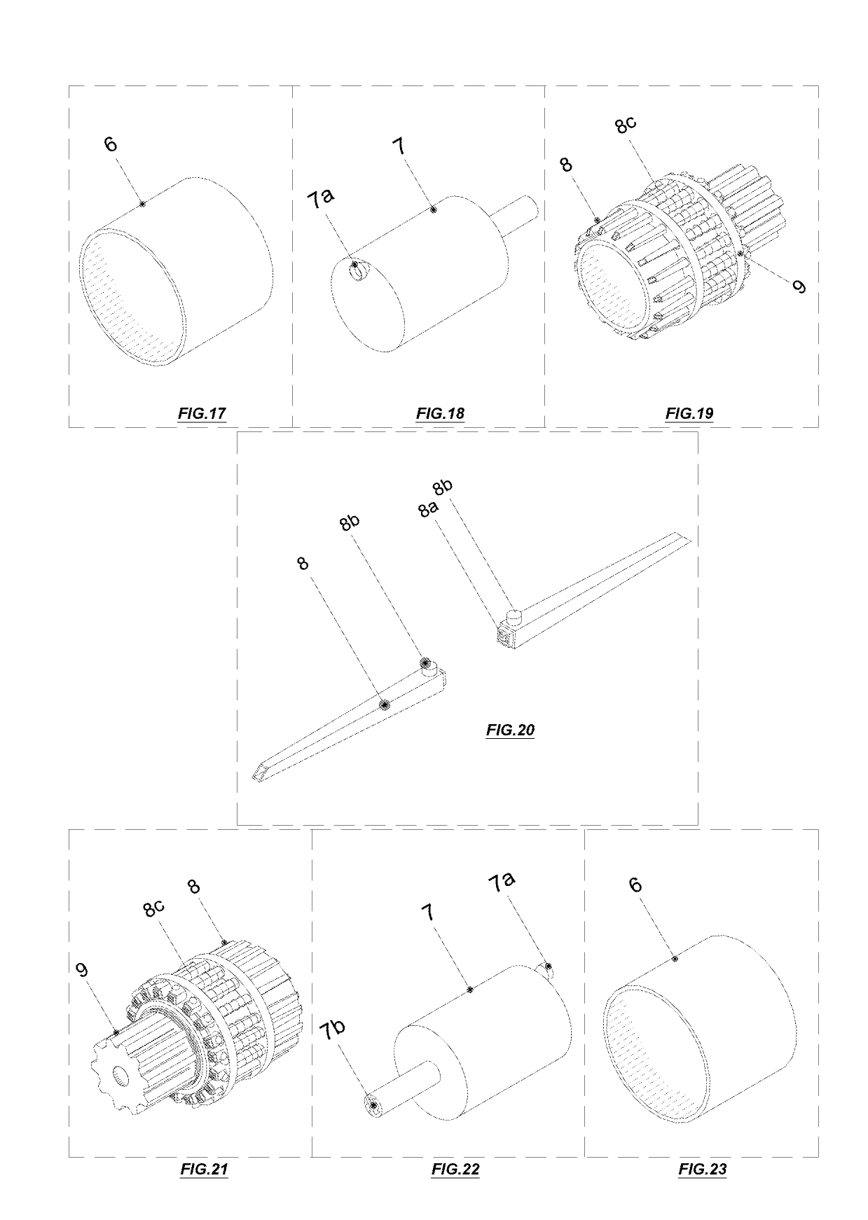

Referring to FIG. 3 is shown that functions as an automatic lancing device, test strip applicator and testing meter. Integrated medical sampling and testing insulin pen device 1 includes pen cap 2 which has an outer plastic cover 4 lancet pricks hole 21, disposal unit of sprit and lancet container 6 attached to the upper pen housing body 3.

The upper pen housing body 3 has contains covering shell 5, touch screen 17 with its control function buttons as menu button 17a, forward button 17b, and back button, main controller button 13, fasting test button 14 for recalculating the blood sugar values as per fasting, normal test button 15 for recalculating the blood sugar values as per normal values, lancet pusher 10 which has in its end microchip test strip 10a, and punching hardness preset gear manual rotator 12a to manually rotate the punching gear 12.

The disposal unit of sprit and lancet container 6 has lancing device which has drum lancets 8, microchip 8a for achieving blood sugar level, lancet griper 8b, and spring 8c for giving smooth movement for lancet while punching, sprit container 7 which has spray nozzle 7a, and sprit sensor chip 7b for making spray for sprit and lancet holder and puncher 9.

Referring to all figures, the lancet pusher 10 has in its end microchip test strip 10a for transferring result collected by the chip 8a in the lancets drum 8, and lancet holder 10b for pushing the lancets 8.

All microchips 8a, 7b, and 10a and all other control and function buttons and the touch screen with its control buttons are controlled and connected to main microcontroller processing unit 16 housed in the housing 3. This microcontroller process and analyte the blood level form the chip 10a and test the blood sugar level and display the result in the touch screen. The result in the touch screen according the test if it in normal or fast case as the button 14, and 15 indicate. The microcontroller controls the pricks force of the lancet according to the patient skin. Microcontroller circuit is power by rechargeable battery 18.

The sampling and testing device 1 has also, USB connection 19 to connect any USB device or flash to update database stored in microprocessor unit RAM or program microprocessor unit again according to current needs. The device 1 has an option to be controlled remotely by smartphone application through the device 1 built-in Bluetooth 20.

To use the device 1 for testing and sampling sugar blood level, we just load the drum of preloaded lancets, control the lancet prick force by rotating the gear 12 to the suitable level, adjust the test type fasting or normal from button 14 and 15, push the lancet pusher 10, and see the result in the screen 17.

For more safe, we must use alcohol for sterilization and disinfection of the lancets 8 by control the chip 11a to pass spray order to microchip 7b for making spray of sprit. There is an electronic plus mechanical gearbox 11 to control the spray in the sprit container 7.

1. An integrated medical sampling and testing insulin pen device has a pen cap 2 attached to a housing 3 wherein the said housing attached to disposal unit of sprit and drum lancets container and the said housing comprises:
contains covering shell;
lancing device
testing meter;
mechanical and electric gearbox;
microcontroller processing unit;
touch screen 17 with its control function buttons as menu button 17a, forward button 17b, and back button;
main controller button 13, fasting test button 14 for recalculating the blood sugar values as per fasting, normal test button 15 for recalculating the blood sugar values as per normal values.

2. According to claim 1, the pen cap has an outer plastic cover 4 and lancet pricks hole 21.

3. According to claim 1, the housing comprises lancet pusher 10 which has in its end microchip test strip 10a, and punching hardness preset gear manual rotator 12a to manually rotate the punching gear 12.

4. According to claim 1, the disposal unit of sprit and drum lancets container has lancing device which has drum lancets 8, microchip 8a for achieving blood sugar level, lancet griper 8b, and spring 8c for giving smooth movement for lancet while punching.

5. According to claims 1, and 4, the said sprit container 7 has spray nozzle 7a, and sprit sensor chip 7b for making spray for sprit and lancet holder and puncher 9.

6. According to claims 1, and 4, the lancet pusher 10 has in its end microchip test strip 10a for transferring result collected by the chip 8a in the lancets drum 8, and lancet holder 10b for pushing the lancets 8.

7. According to claim 1, all microchips 8a, 7b, and 10a and all other control and function buttons and the touch screen with its control buttons are controlled and connected to main microcontroller processing unit 16 housed in the housing 3.

8. An integrated lancing device with testing meter comprises:
housing comprises: covering shell; lancing device, testing meter, mechanical and electric gearbox, microcontroller processing unit, touch screen 17 with its control function buttons as menu button 17a, forward button 17b, and back button, main controller button 13, fasting test button 14 for recalculating the blood sugar values as per fasting, normal test button 15 for recalculating the blood sugar values as per normal values, USB, and built-in Bluetooth 20 to connect smartphone application through the said device 1.
pen cap has an outer plastic cover 4 and lancet pricks hole 21 wherein the pen cap attached to the said housing;
disposal unit of sprit and drum lancets container has lancing device which has drum lancets 8, microchip 8a for achieving blood sugar level, lancet griper 8b, and spring 8c for giving smooth movement for lancet while punching and sprit container 7 has spray nozzle 7a, and sprit sensor chip 7b for making spray for sprit and lancet holder and puncher 9.

9. According to claim 9, the housing comprises lancet pusher 10 which has in its end microchip test strip 10a, and punching hardness preset gear manual rotator 12a to manually rotate the punching gear 12.

10. According to claims 1, and 8, the microcontroller process and analyte the blood level form the chip 10a and test the blood sugar level and display the result in the touch screen wherein the microcontroller controls the pricks force of the lancet according to the patient skin and the said microcontroller circuit is power by rechargeable battery 18.

11. According to all above claims, alcohol used for sterilization and disinfection of the lancets 8 by control the chip 11a to pass spray order to microchip 7b for making spray of sprit wherein an electronic plus mechanical gearbox 11 to control the spray in the sprit container 7.

12. Method for sampling and testing blood sugar level comprise:
load the drum of preloaded lancets;
control the lancet prick force by rotating the gear 12 to the suitable level;
adjust the test type fasting or normal from button 14 and 15;
use alcohol for sterilization and disinfection of the lancets 8 by control the chip 11a to pass spray order to microchip 7b for making spray of sprit wherein the electronic plus mechanical gearbox 11 control the spray in the sprit container 7 push the lancet pusher 10;
see the result in the screen 17.

13. According to claim 11, the method to sampling and testing the sugar blood level using the multiclix integrated sampling and testing meter pen in claim 1, and claim 8.
